(19) United States
(12) Patent Application Publication
(10) Pub. No.: US 2013/0071426 A1
(43) Pub. Date: Mar. 21, 2013

(54) TOPICAL PHARMACEUTICAL OR COSMETIC COMPOSITION USEFUL FOR THE TREATMENT OF DISEASES OR CONDITIONS THAT TRANSCUR THROUGH A DEFICIT OF MATURATION OF THE CORNIFIED ENVELOPE

(71) Applicants: Noemí Serra-Baldrich, Barcelona (ES); Silvia García Bertran, Barcelona (ES)

(72) Inventors: Noemí Serra-Baldrich, Barcelona (ES); Silvia García Bertran, Barcelona (ES)

(73) Assignee: LABORATORIOS LETI, S.L., BARCELONA (ES)

(21) Appl. No.: 13/700,013

(22) PCT Filed: May 26, 2011
(86) PCT No.: PCT/EP11/58674

§ 371 Date: Nov. 26, 2012

(30) Foreign Application Priority Data
May 27, 2010 (EP) 10382147.6

(51) Int. Cl. A61K 36/03; A61K 8/49; A61K 8/96; A61K 8/34; A61K 31/047; A61K 31/455

(52) U.S. Cl. CPC A61K 36/03; A61K 31/047; A61K 31/455; A61K 8/96; A61K 8/345; A61K 8/4926
USPC 424/195.17

(57) ABSTRACT

It comprises a combination of glycerin, niacinamide, and an extract of Fucus Serratus, to topical pharmaceutical or cosmetic compositions containing them, processes for their preparations, as well as their pharmaceutical use in the prophylaxis and/or treatment of disease or conditions that transcur through a deficit of maturation of the cornified envelope, and their cosmetical use as a skin care agent, skin barrier recovery agent, and moisturizer.

The present invention relates to the field of pharmacy and cosmetics, in particular, it relates to a combination of active ingredients, to topical compositions containing them, and their pharmaceutical use for the prophylaxis and/or treatment of disease or conditions that transcur through a deficit of maturation of the cornified envelope, and their cosmetical use as a skin care agent, skin barrier recovery agent, and moisturizer.

### BACKGROUND ART

The stratum corneum (SC) is the most external layer of the epidermis. It contains terminally differentiated keratinocytes (corneocytes) and intercellular lipids surrounding them. Corneocyte cells are lined with a 15 nm thick layer of proteins cross-linked by isopeptide and disulfide bonds, called the cornified envelope (CE). A portion of omega-hydroxyceramides (ω-OH Cer) is diverted to the external surface of the cornified envelope (CE), where it is attached covalently to involucrin and other constituents of the CE through a transglutaminase 1 activity. The resulting monolayer of ω-OH Cer forms the cornified lipid envelope (CLE). This structure gives resistance and hydrophobicity to the corneocyte. The CE is a thin and rigid insoluble structure enveloping corneocytes, and is one of the most important structures for maintaining the barrier function in the SC.

The CE is formed by complex processes. During the terminal differentiation of epidermal keratinocytes, called cornified maturation, one of the initial events is the expression of CE precursor proteins. These CE precursor proteins, including involucrin, loricrin, and small proline-rich proteins, among others, are cross-linked by transglutaminase enzymes. Another important event is the acquisition of hydrophobicity by covalent attachment to the lipids, mainly omega-hydroxyceramides, to the extra cellular surface of CE components.

It has been reported the existence of the following distinct classes of cornified envelopes: the polygonal rigid cornified envelope corneocytes (CEr) or mature CEr; and the irregularly shaped fragile cornified envelope corneocytes (CEf) or immature CEf. The immature CEf are characterized by high involucrin antigenicity and less hydrophobicity. The immature CEf are normally found only in the deeper layer of the SC, whereas upper layers of the SC consist essentially of mature CEr, suggesting a suitable maturation process of the cornified envelope.

In some skin disease or condition including psoriasis, atopic dermatitis, lamellar ichthyosis, contact dermatitis, skin aging, and cutaneous xerosis, it have been identified, or have been presupposed, the appearance of immature CEf, not only in the deeper layers of the SC but also in the outer layers of the SC. The presence of these immature CEf in the outer layers of the SC is associated with a poor barrier function (or parakeratosis), suggesting an abnormal and/or incomplete maturation process of the corneocytes, and/or a reduction of transglutaminase activity, and/or a reduction of corneocyte envelope cross-linking events, and/or an increase in the transepidermal water loss (TEWL).

In particular, xerosis (or dry skin) is a common dermatosis or condition of high prevalence in the general population affecting people of varying skin types and ages and various areas of the body. It is clinically characterized by skin roughness, scaliness, and pruritus. The skin shows the tendency to crack, causing deep fissures in cases of extremely dry skin. It is considered dry skin when the water content of the SC is below 10%. While the pathophysiology of this process is complex, disruption of normal epidermal differentiation is one of the principal etiologic factors. This disturbance gives rise to changes in the fragile corneocyte envelope (CEf), and rigid corneocyte envelope (CEr) level, where CEf predominate and to imbalance in water content, impairing barrier function which leads a reduction in intercellular lipid and protein content of the SC.

Thus, the role of the CE, its constituent proteins, and its transglutaminase-mediated maturation processes has been shown to be essential for a good skin condition. Thus, corneodesmolysis, the enzyme mediated degradation of inter-corneocyte linking structures responsible for the regulation of the shedding of corneocytes at the surface of the skin, is reduced in the disorders or conditions as mentioned above. The reduction of corneodesmolysis is caused by the reduction in the levels, and activities of SC proteases, together with elevated levels of corneodesmosomal glycoproteins in the superficial layers of the SC.

The treatment of the above mentioned skin diseases or conditions wherein immature CEf are observed at the outer layers of the SC accompanied by parakeratosis, for instance, diseases or conditions that transcur with dry skin, are mainly based by the external application of anti-inflammatory agents including corticosteroids. Disadvantages of these agents are related to their mineralocorticoid effect and their antiproliferative activity against human epidermal keratinocytes and fibroblasts, providing a decrease in the thickness of dermis and epidermis. Another undesirable effect of corticosteroid treatment is their effect against the local microbial flora provoking the appearance of dermatitis, rosacea, folliculitis, pruritus, and acne eruptions.

An alternative treatment is the use of active components which promote the maturation of the CE of the corneocytes of the SC, which is associated with the restoration of the skin barrier function. In particular, the European patent application EP 1374832 discloses skin care cosmetics which comprise effective amount of moisturizers, antioxidant and mineral salts which have the efficacy to promote the maturation of CE for ameliorating rough skin.

Besides, the European patent application EP 1618867 discloses the use of skin care cosmetic compositions which comprise a moisturizer, preferably glycerin, a vitamin, preferably niacinamide, for breaking the cycle of dry skin.

From what is known in the art it is derived that there is still the need of providing more effective promoting agents in the maturation of the corneocytes and the restoration of the skin barrier function.

### SUMMARY OF THE INVENTION

Inventors have found that a combination of glycerin, niacinamide, and an extract of Fucus Serratus promotes a higher maturation of CE of the corneocytes of the SC than the one that could be expected due to a synergistic effect.

It is known in the state of the art that glycerin is involved in the process of corneocyte maturation promoting the activation of residual transglutaminase activity retained within the SC (Cf. A. V. Rawlings et al. “Moisturizer technology versus clinical performance”. Dermatologic Therapy, 2004, vol. 17, pp. 49-56). It is also known that niacinamide (nicotinamide, vitamin B3 or vitamin PP) increases the synthesis of certain CE precursor proteins including involucrin, filagrin and keratin 1, the production of ceramides in human keratinocytes, and the increase of activity of transglutaminases. The improvement in the amount of these proteins together with the increase in the novo synthesis of some lipids promotes the differentiation of keratinocytes and restores the skin barrier function (Cf. N. Kitamura et al. “effect of niacinamide on the differentiation of human keratinocyte”, Journal of Dermatological Science, 1996, vol. 12, pp. 202). Likewise it has been also reported that extracts of algae, including the brown seaweed Fucus Serratus improves skin barrier function because of the promotion of the maturation of corneocytes by the increase of transglutaminase activity. However, neither a combination of these three components has ever been suggested nor its synergistic effect in the promotion of the maturation of CE of the corneocytes of the SC.

Thus, an aspect of the present invention refers to a combination of glycerin, niacinamide, and an extract of Fucus Serratus. As it is illustrated in the example 2, the promoting effect in the corneocyte maturation shown for the combination of the active ingredients of the present invention is higher than the promoting effect of a mixture of glycerin and niacinamide, and an extract of Fucus Serratus separately, and also higher than the expected effect of their combination.

Another aspect of the present invention refers to a topical pharmaceutical or cosmetic composition which comprises an effective amount of the combination as defined above together with one or more appropriate topical pharmaceutically or cosmetically acceptable excipients or carriers.

Another aspect of the present invention refers to the pharmaceutical composition as defined above, for use in the prophylaxis and/or treatment of a disease or condition which transcur through a deficit of maturation of the cornified envelope, wherein the disease or condition is selected from the group consisting of cutaneous xerosis, psoriasis, lamellar ichthyosis, skin aging, contact dermatitis, and atopic dermatitis.

Finally, another aspect of the present invention refers to the use of the cosmetic composition as defined above as a skin care agent, where the skin care comprises ameliorating at least one of the following symptoms: roughness, flakiness, dehydration, tightness, chapping, and lack of elasticity.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

The term “weight ratio” refers to the relation of weights of glycerin, niacinamide and the extract of Fucus Serratus needed to promote the maturation of corneocytes.

The term “percentage (%) by weight” refers to the percentage of each ingredient of the combination or composition in relation to the total weight.

An “effective amount” of the combination refers to the amount of active ingredients which provide a therapeutic or cosmetic effect after its application.

The term “pharmaceutically acceptable” refers to that excipients or carriers suitable for use in the pharmaceutical technology for preparing compositions with medical use.

The term “cosmetically acceptable” or “dermatological acceptable” which is herein used interchangeably refers to that excipients or carriers suitable for use in contact with human skin without undue toxicity, incompatibility, instability, allergic response, among others.

The term “hydrating agent” or “moisturizer” or “moisturizing agent” which is herein used interchangeably refers to a material which increases the water content of the skin and helps keep it soft and smooth.

The term “skin barrier recovery agent” refers to material whose composition and/or structure are similar to the skin barrier allowing the reparation of its deficiencies.

The term “emollient” agent refers to a material that softens and soothes the skin in order to correct dryness and scaling of the skin, lubricating the skin surface, encouraging skin water retention, and altering product textures.

The term “humectant” agent refers to a hygroscopic material which attracts water molecules from the surrounding environment though either absorption or adsorption, preventing the skin from losing moisture.

The term “thickening agent” or “thickener” or “viscosity agent” which is herein used interchangeably refers to a material that increases its viscosity without substantially modifying its other properties.

The term “emulsifying agent” or “emulsifier” which is herein used interchangeably refers to a material that reduces surface tension, promoting the formation of intimate mixtures of non-miscible liquids by altering the interfacial tension. Emulsifier stabilizes an emulsion by increasing its kinetic stability.

The term “surfactant” refers to a material which lowers the surface tension of a liquid and the interfacial tension between two liquids, allowing their easier spreading. Surfactants have a hydrophilic head that is attracted to water molecules and a hydrophobic tail that repels water and simultaneously attaches itself to oil and grease in dirt. These opposing forces loosen the dirt and suspend it in the water, having the ability to remove it from surfaces such as the human skin, textiles, and other solids, when surfactants are dissolved in water.

The term “antioxidant” refers to a material that slows or prevents the oxidation of other molecules. Antioxidants include free radical scavengers and reducing agents.

The term “pH-regulating” agent refers to acids or bases that can be used to adjust the pH of the finished product to the desired level, without affecting the stability of the solution.

The term “preservative” refers to a material that prevents or reduces or slows down microbial growth, providing that the stability of the solution is not affected.

The term “surfactant base” or “surfactant system” which is herein used interchangeably refers to a blend of surfactants, preferably anionic and amphoteric surfactants, which tends to form spherical micelles which are isotropic with low viscosity, or tends to form liquid crystalline phases of hexagonal and lamellar phases, which are anisotropic with higher viscosity. Spherical micelles are preferred for the preparation of shampoos and body or face washes.

The term “hydrophilic” solvent refers to solvents that are capable of creating hydrogen bonding, enabling them to be dissolved more readily in water, and in other polar solvents.

The term “lipophilic” solvent refers to non-polar solvents that have little or no capacity to form hydrogen bonds, enabling them to be dissolved in fats, oils, lipids, and other non-polar solvents.

The term “glycol” or “vicinal diol” or “1,2-diols” which is herein used interchangeably refers to aliphatic organic compounds in which two hydroxyl (OH) groups are attached to adjacent carbon atoms.

The term “extract” of Fucus Serratus refers to the conventional sense to refer to concentrated preparations of the algae obtained by removing the active constituents from the algae with suitable means. Such actives constituents can be obtained from various parts of algae. Suitable means for removal of the active ingredients include, for example, use of organic solvents, microwave or supercritical fluids extraction. Active ingredients are sometimes directly incorporated in food, pharmaceutical or cosmetic compositions in a variety of forms, including a pure or semi-pure component, a solid or liquid extract, or a solid algae matter. Algae extracts contain not only one but multiple constituents, many of them active. Often, the beneficial effect is derived from the combination of many of these active compounds, even though in some cases there is one particular compound that is mainly responsible for most of the activity.

As mentioned above, an aspect of the present invention refers to a combination which comprises glycerin, niacinamide, and an extract of Fucus Serratus.

As it is illustrated in the example 2, the combination of glycerin, niacinamide, and an extract of Fucus Serratus have a synergistic effect in promoting the maturation of the corneocytes allowing a reduction in the number of immature CEf in the outer layer of the SC.

The extract of Fucus Serratus is selected from a hydrosoluble or liposoluble extract. The use of hydrophilic or lipophilic solvents respectively allow the extraction from the algae of the active compounds which are effective for promoting the maturation of the corneocytes of the SC. Hydrophilic solvents suitable for the preparation of the extract of Fucus Serratus of the present invention include glycols. Examples of suitable glycols are selected from the group consisting of ethylene glycol, propylene glycol, butylene glycol, diethylene glycol, tetramethylene glycol, or 1,2,3-propanetriol (glycerin). Lipophilic solvents suitable for the preparation of the extract of Fucus Serratus of the present invention are selected from the group consisting of caprylic/capric triglyceride, vegetable oils including Helianthus Annuus seed oil, mineral oils, animal fats, their fractions and mixtures.

In a preferred embodiment the extract of Fucus Serratus is a glycolic extract where when the glycolic extract of Fucus Serratus is a glycerol extract, then the extract contains a part or the total amount of the glycerin of the combination.

In another preferred embodiment the glycolic extract of Fucus Serratus is a glycerol extract. When the glycerol extract of Fucus Serratus is used in the combination of the present invention, then the extract contains a part or the total amount of the glycerin of the combination.

In a preferred embodiment, when the glycolic extract is a glycerol extract, then the extract contains the total amount of the glycerin of the combination. And, in another preferred embodiment, when the glycolic extract is a glycerol extract, then the extract contains a part of the amount of the glycerin of the combination (cf. Example 1).

In a preferred embodiment, the weight ratio between glycerin, niacinamide, and the glycolic extract of Fucus Serratus is comprised between 1.5:1:0.1 and 1.5:1:0.5. In another preferred embodiment, the weight ratio of the active ingredient is comprised between 1.5:1:0.2 and 1.5:1:0.4. Preferably, the mentioned ratio is 1.5:1.0:0.2. As it is shown in the examples, especially good activity as a promoting agent of the maturation of the cornified maturation process is obtained with the combination of the active ingredients of the present invention.

This aspect could be also formulated as a weight percent of glycerin, niacinamide, and the glycolic extract of Fucus Serratus in the combination of the present invention.

Thus, the weight percent of the active ingredient is comprised between:

50-57.7% by weight of glycerin;

33.3-38.4% by weight of niacinamide; and

3.8-16.6% by weight of the glycerol extract of Fucus Serratus,

being the sum of the active ingredients 100%.

In a preferred embodiment, the above-mentioned weight percentage is:

55.5% by weight of glycerin;

37.0% by weight of niacinamide; and

7.4% by weight of the glycerol extract of Fucus Serratus.

In a particular embodiment, the weight ratio of glycerin and niacinamide can vary. Particularly, the weight ratio is comprised between 1.5:1 and 10:1. Preferably, the mentioned ratio is comprised between 2.8:1 and 10:1.

The combination of the present invention can be in form of a topical pharmaceutical or cosmetic composition. Thus, the topical pharmaceutical or cosmetic composition of the present invention comprises an effective amount of the combination as defined above together with one or more appropriate topical pharmaceutically or cosmetically acceptable excipients or carriers.

In a particular embodiment, the topical composition is a pharmaceutical composition comprising an effective amount of the combination as defined above together with one or more appropriate topical pharmaceutically acceptable excipients or carriers.

In another particular embodiment, the topical composition is a cosmetic composition comprising an effective amount of the combination as defined above together with one or more appropriate topical cosmetically acceptable excipients or carriers.

The topical compositions defined above comprise appropriate excipients or carriers for topical administration that can be pharmaceutical or cosmetic excipients, including, but not limited to, repairing cutaneous barrier function agent, a hydrating agent, an emollient, an emulsifier, a thickener, a humectant, a pH-regulating agent, an antioxidant, a preservative agent, a vehicle, or their mixtures. The excipients or carriers used have affinity for the skin, are well tolerated, stable, and are used in an amount adequate to provide the desired consistency, and ease application.

Examples of appropriate topical skin barrier recovery agent include, but are not limited to, ceramides, cholesterol, fatty acids, and precursors of these lipids including cerebrosides, sphingoid bases such as phytosphingosine or sphingosine, or phospholipids including phosphatidylcholine, and agents that promote the synthesis of epidermal lipids like urea, dexpanthenol, and alpha-hydroxyacids including lactic acid among others. Preferably, ceramides are selected from the group consisting of ceramide 1, ceramide 3, and ceramide 6 II. The amount of skin barrier recovery agent in the compositions of the present invention is comprised between 0.05 and 10%.

Examples of appropriate topical hydrating agent include, but are not limited to, collagen, collagen amino acids, dimethiconol, glycine, hyaluronic acid, dimethylsilanol hyaluronate, magnesium stearate, maltitol, maltose, pyrrolidone carboxylic acid (PCA), manganese PCA, sodium PCA, mannitol, trehalose, trilactin, glucose, glutamic acid, hydrolyzed caesalpinia spinosa gum, caesalpinia spinosa gum, prunus persica extract, prunus serotina extract, echinacea angustifolia extract, Echinacea purpurea extract, methyl gluceth, hydrolyzed wheat gluten, erythritol, aluminium stearoyl glutamate, copper acetylmethionate, or ditridecyl dimmer dilinoleate. Preferably the hydrating agent is selected from the group consisting of glucose, glycine, lysine, glutamic acid, hydrolyzed caesalpinia spinosa gum, caesalpinia spinosa gum, sodium PCA, and their mixtures. The amount of hydrating agent in the compositions of the present invention is comprised between 0.1 and 15%.

Examples of appropriate topical emollient agents include, but are not limited to, octyl hydroxystearate, lanolin, caprylic/capric triglyceride, cetyl palmitate, octyldodecanol, cetyl alcohol, isopropyl isostearate, glyceryl dilaurate, isopropyl myristate, palm alcohol, dimethicone, squalane, plukenetia volubilis seed oil, butyrospermum parkii butter, sucrose cocoate, or their mixtures. Preferably the emollient is selected from the group consisting of dimethicone, squalane, plukenetia volubilis seed oil, butyrospermum parkii butter, caprylic/capric triglyceride, octyldodecanol, or their mixtures. The amount of emollient agent in the compositions of the present invention is comprised between 10 and 30%.

Examples of appropriate emulsifier include, but are not limited to, glyceryl trioleate, glyceryl oleate, acetylated sucrose distearate, sorbitan trioleate, polyoxyethylene monostearate, glycerol monooleate, sucrose distearate, polyethylene glycol monostearate, octyl phenoxypoly (ethyleneoxy) ethanol, deacylerin penta-isostearate, sorbitan sesquioleate, hydroxylated lanolin, lecithin, lanolin, triglyceryl diisostearate, polyoxyethylene oleyl ether, calcium stearoyl-2-lactylate, sodium lauroyl lactylate, sodium stearoyl lactylate, cetearyl glucoside, methyl glucoside sesquistearate, sorbitan monopalmitate, methoxy polyethylene glycol-22/dodecyl glycol copolymer, polyethylene glycol-45/dodecyl glycol copolymer, polyethylene glycol 400 distearate and glyceryl stearate, candelilla/jojoba/rice bran polyglyceryl-3 esters, cetyl phosphate, potassium cetyl phosphate, or their mixtures. Preferably, the emulsifier is selected group consisting of glyceryl oleate, lecithin, sodium lauroyl lactylate, sodium stearoyl lactylate, glyceryl stearate, candelilla/jojoba/rice bran polyglyceryl-3 esters, and their mixtures. The amount of the emulsifier in the compositions of the present invention is comprised between 0.5 and 10%.

Examples of appropriate surfactant agents include, but are not limited to, non-ionic, ionic (either anionic or cationic) or zwitterionic (or amphoteric wherein the head of the surfactant contains two oppositely charged groups) surfactants. Examples of anionic surfactants include, but are not limited to, those based on sulfate, sulfonate or carboxylate anions such as perfluorooctanoate (PFOA or PFO), alkyl benzene sulfonate, soaps, fatty acid salts, or alkyl sulfate salts such as perfluorooctanesulfonate (PFOS), sodium dodecyl sulfate (SDS), ammonium lauryl sulfate, or sodium lauryl ether sulfate (SLES). Examples of cationic surfactants include, but are not limited to, those based on quaternary ammonium cations such as or alkyltrimethylammonium including cetyl trimethylammonium bromide (CTAB) a.k.a., or hexadecyl trimethyl ammonium bromide, cetylpyridinium chloride (CPC), polyethoxylated tallow amine (POEA), benzalkonium chloride (BAC), or benzethonium chloride (BZT). Examples of zwitterionic surfactants include, but are not limited to dodecyl betaine, cocamidopropyl betaine, or coco ampho glycinate. Examples of non-ionic surfactants include, but are not limited to, alkyl poly(ethylene oxide), alkylphenol poly(ethylene oxide), copolymers of poly(ethylene oxide), poly(propylene oxide) (commercially called Poloxamers or Poloxamines), alkyl polyglucosides including octyl glucoside and decyl maltoside, fatty alcohols including cetyl alcohol and oleyl alcohol, cocamide MEA, cocamide DEA, or polysorbates including tween 20, tween 80, or dodecyl dimethylamine oxide. Preferably, the surfactant is foaming and skin friendly, including polysorbate 20 or 40, coco glucoside, lauryl glucoside, decyl glucoside, lauryl sulfates such as ammonium, sodium, magnesium, MEA, triethylamine (TEA), or mipa lauryl sulfate, cocamidopropyl betain, or sodium alkyl sulfosuccinates. The amount of the surfactant in the compositions of the present invention is comprised between 0.5 and 10%.

Examples of appropriate topical humectants include, but are not limited to, glycerin, diglycerin, ethylhexylglycerin, glucose, honey, lactic acid, polyethylene glycol, propylene glycol, sorbitol, sucrose, or threalose. Preferably, the humectant is selected group consisting of glycerin, diglycerin, ethylhexylglycerin, and their mixtures. The amount of the humectants in the compositions of the present invention is comprised between 0.5-10%.

Examples of appropriate topical pH-regulating agents include, but are not limited to, acetic acid, lactic acid, citric acid, ethanolamine, formic acid, oxalic acid, potassium hydroxide, sodium hydroxide, triethanolamine, or their mixtures. Preferably, the pH-regulating agent is selected group consisting of triethanolamine, sodium hydroxide, lactic acid, and citric acid. The amount of the pH-regulating agent in the compositions of the present invention is comprised between 0.01 and 1%.

Examples of appropriate antioxidants include, but are not limited to, free radical scavengers or reducing agents such as, acetyl cysteine, ascorbic acid, ascorbyl palmitate, butylated hydroxytoluene, green tea extract, caffeic acid, cysteine, tocopherol, ubiquinone, propyl gallate, butylated hydroxytoluene (BHT), and their mixtures. Preferably, the antioxidant agent is selected group consisting of ascorbyl palmitate, and tocopherol. The amount of the antioxidants in the compositions of the present invention is comprised between 0.001 and 0.25%.

Examples of appropriate preservative agents include, but are not limited to, benzoic acid, butylparaben, ethylparaben, propylparaben, methylparaben, sorbic acid, potassium sorbate, sodium benzoate, phenoxyethanol, triclosan, or their mixtures. Preferably, the preservative agent is selected group consisting of potassium sorbate, sodium benzoate, and phenoxyethanol. The amount of the preservatives in the compositions of the present invention is comprised between 0.1 and 3%.

Examples of appropriate viscosity agents include, but are not limited to, cellulose or their derivatives such as hydroxypropyl methylcellulose, polyethylene glycol, microcrystalline cellulose, cetearyl alcohol, alginates, branched polysaccharides, fumed silica, xanthan gum, carbomer, and polyacrylates. Preferably, the viscosity agent is selected group consisting of microcrystalline cellulose, cetearyl alcohol, cellulose, xanthan gum, and carbomer. The amount of the viscosity agents in the compositions of the present invention is comprised between 0.5 and 10%.

The compositions mentioned above also include a vehicle. Examples of vehicles include, but are not limited to, water, propylene glycol, butylene glycol, ethanol, isopropanol, or silicones. Preferably, the vehicle is water.

Additionally, the compositions of the present invention may contain other ingredients, such as fragrances, colorants, and other components known in the state of the art for use in topical formulations.

The topical compositions of the invention can be formulated in several forms that include, but are not limited to, solutions, aerosols and non-aerosol sprays, shaving creams, powders, mousses, lotions, gels, sticks, ointments, pastes, creams, shampoos, shower gel, body washes or face washes.

The topical composition used is formulated preferably as an emulsion. An emulsion is a dispersed system comprising at least two immiscible phases, one phase dispersed in the other as droplets. The above mentioned emulsifying agents are included to improve stability. When water is the dispersed phase and oil is the dispersion medium, the emulsion is termed a water-in-oil emulsion (w/o). When oil is dispersed as droplets throughout the aqueous phase, the emulsion is termed an oil-in-water emulsion (o/w). Other types of emulsions known in the art are multiple emulsions, such as water-in-oil-in-water emulsions (w/o/w), GELTRAP emulsions, where the aqueous intern phase is gelified and it is covered by the oil phase, and SWOP emulsions, also known as inversion emulsions. The emulsions used are preferably oil-in-water emulsions. Preferably, the emulsions for use in the sense of the present invention are compatible with creams and lotions.

Another topical composition used is formulated preferably as a “surfactant base”. A surfactant base is a blend of at least two surfactants. Surfactants are commonly used in cleaning products, breaking up stains and keeping the dirt in the water solution to prevent its re-deposition onto the surface. Surfactants disperse dirt that normally does not dissolve in water, becoming it dispersible in water, and removable with the wash water. The above mentioned surfactants are included to lower the surface tension. Preferably, surfactant bases for use in the sense of the present invention are compatible with shampoos, shower gel, and body or face washes.

Topical compositions of the present invention can be prepared according to methods well known in the state of the art. The appropriate excipients and/or carriers, and their amounts, can readily be determined by those skilled in the art according to the type of formulation being prepared.

The topical pharmaceutical composition of the invention can be used for topical application to the skin for promoting the maturation of the corneocytes. Thus, another aspect of the present invention is a topical pharmaceutical composition as defined above, for use in the prophylaxis and/or treatment of a disease or condition which transcur through a deficit of maturation of the cornified envelope, wherein the disease or condition is selected from the group consisting of cutaneous xerosis, psoriasis, lamellar ichthyosis, skin aging, contact dermatitis, and atopic dermatitis. This aspect could be also formulated as the use of the topical pharmaceutical composition as defined above for the preparation of a medicament for the prophylaxis and/or treatment of a disease or condition which transcur through a deficit of maturation of the cornified envelope, wherein the disease or condition is selected from the group consisting of cutaneous xerosis, psoriasis, lamellar ichthyosis, skin aging, contact dermatitis, and atopic dermatitis. It also relates to a method for the prophylaxis and/or treatment of a mammal suffering or is susceptible to suffer from a disease or condition which transcur through a deficit of maturation of the cornified envelope, wherein the disease or condition is selected from the group consisting of cutaneous xerosis, psoriasis, lamellar ichthyosis, skin aging, contact dermatitis, and atopic dermatitis, the method comprises administering to said mammal an effective amount of the topical pharmaceutical composition of the present invention. Thus, the promoting effect in the cornified envelope maturation of the combination of the present invention is shown in the results of Example 2.

The above-mentioned skin disorders or conditions that transcur with the appearance of immature CEf in the upper layers of the SC accompanied with a decrease of the number of mature CEf in the SC. These conditions have been associated with a deficit in the epidermal differentiation. In particular the appearance of immature CEf in the outer layers of the skin indicates an incomplete or alteration of the final step of the epidermal differentiation, that is cornified envelope maturation. (cf. A. V. Rawlings. “Trends in stratum corneum research and the management of dry skin conditions”. International Journal of Cosmetic Science. 2003, vol. 25, pp. 63-95; A. V. Rawlings. “Stratum corneum moisturization at the molecular level: an update in relation to the dry skin cycle”, The Journal of Investigative Dermatology, 2005, vol. 124, pp. 1099-1110; Hirao T. et al. “A novel non-invasive evaluation method of cornified envelope maturation in the stratum corneum provides a new insight for skin care cosmetics”. IFSCC Magazine, 2003, vol. 6, pp. 103; Halzle E. “Effects of dermatitis, stripping, and steroids on the morphology of corneocytes. A new bioassay”, The journal of investigate dermatology, 1977, vol. 68(6), pp. 350-6; Jane Fore-Pliger. “The epidermal skin barrier: Implications for the Wound Care Practitioner, Part I”, Advances in Skin Wound Care, 2004, vol. 17, pp. 417; and Contet-Audonneau J L, “A histological study of human wrinkle structures: comparison between sun-exposed areas of the face, with or without wrinkles, and sun-protected areas”, The British journal of dermatology, 1999, vol. 140(6), pp. 1038-47).

Thus, in a preferred embodiment, it is provided the topical pharmaceutical composition as defined above for use in the prophylaxis and/or treatment of cutaneous xerosis (dry skin).

The cosmetic composition of the invention can be used for the care of the skin. Thus, another aspect of the present invention is a use of the topical cosmetic composition as defined above for the skin care. In a preferred embodiment, the cosmetic compositions of the present invention are used for the skin care, where the skin care comprises ameliorating at least one of the following symptoms: roughness, flakiness, dehydration, tightness, chapping, and lack of elasticity.

In another aspect of the invention, the cosmetic composition as defined above are used as a skin care agent, wherein the skin care comprises ameliorating at least one of the following symptoms: roughness, flakiness, dehydration, tightness, chapping, and lack of elasticity.

The topical cosmetic composition of the present invention is designed to apply to the body to improve its appearance or to beautify, preserve, condition, cleanse, color or protect the skin, nails or hair (cf. Academic press Dictionary of Science and Technology, 1992, pp. 531; A terminological Dictionary of the Pharmaceutical Sciences. 2007, pp. 190). Therefore, the above cosmetic compositions are adjectivally used for a non-medical application.

In a preferred embodiment the topical cosmetic compositions of the present invention is a moisture agent. In another preferred embodiment the topical cosmetic compositions of the invention is a skin care agent. In another preferred embodiment the topical cosmetic compositions of the invention is a soothing agent. Thus, it is also part of the invention the use of the topical cosmetic composition as defined above as a soothing agent. The soothing agent is suitable for allaying, calming, composing, lulling, quieting, settling, stilling, or tranquilizing the skin.

In a preferred embodiment, the cosmetic composition of the invention can be used for topical application to the skin for restoring the skin barrier function. Thus, it is provided the use of the topical cosmetic composition as defined above as a skin barrier recovery agent.

It has been reported that the appearance of less hydrophobic immature CEf in the outer layers of the SC is associated with an alteration of the skin barrier function due to the acquired superficial position of these immature CEf (cf. T. Hirao, et al. “Identification of immature cornified envelopes in the barrier-impaired epidermis by characterization of their hydrophobicity and antigenicity of the components”, Experimental Dermatology, 2001, Vol. 10, pp. 35-44). The change in the structure of the epidermis is accompanied by an increase in the transepidermal water loss (TEWL) levels. TEWL is the amount of water which is evaporated per hour, and square centimeter of skin, and this parameter is used for identifying the state of the skin barrier. An increased in TEWL means that exist a certain level of barrier damage due to a lack of barrier substances, and, as a consequence, the skin is drying out due to humidity loss.

The topical cosmetic composition which comprises the combination of the present invention promotes the cornified envelope maturation of the immature CEf, reduces the TEWL levels, and restores the lipid barrier of the skin. Thus, the re-establishment of the above parameters contributes to the restoration of the skin barrier function.

In a preferred embodiment the above cosmetic compositions are used as a moisturizing agent. It is known that mature corneocytes have a high capacity to retain water because of their high hydrophobicity, and rigidity, due to the presence of the mature cornified envelope, and the present of the natural moisturizing factor (NMF) located inside of the corneocytes. The NMF is a mixture of hydrosoluble and/or hydrodispersable molecules formed as a result of the degradation of filagrin protein during the cornified maturation, allowing the retention of water into the mature corneocytes.

It is known that when the cornified envelope maturation is altered the content of water of the SC and the amount of NMF inside of the corneocytes are reduced. The presence of moisturizing agents in the composition of the present invention, and the synergistic effect in the promotion of the cornified envelope maturation of CEf by the combination of the present invention, allows the restoration of the skin barrier function and the re-establishment of the appropriate hydration level of the skin.

The cosmetic composition of the present invention can be used as a skin care agent, where the skin care comprises ameliorating at least one of the following symptoms: roughness, flakiness, dehydration, tightness, chapping, and lack of elasticity. These symptoms are generally caused by microorganisms and other substances that can easily penetrate into the skin causing irritations, allergies and inflammations, or by certain environmental conditions, including humidity, temperature, the time of year (season variation), and the moisture content of the skin (hydration level). Besides, these symptoms may also be associated with a disease or condition selected from the group consisting of cutaneous xerosis, psoriasis, lamellar ichthyosis, skin aging, contact dermatitis, and atopic dermatitis.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples are provided by way of illustration, and they are not intended to be limiting of the present invention. Furthermore, the present invention covers all possible combinations of particular and preferred embodiments described herein.

### EXAMPLES

The following abbreviations are used in the below examples:

PEG: polyethylene glycol

EDTA: ethylene diamine tetra ammonium

The technical specifications of the Epiderm® tissue supplied by MatTek corporation are summarized as follow:

I. Cells:
type: normal human epidermal keratinocytes (NHEK)
derived from: neonatal-foreskin tissue

II. Medium:
Base medium: Dulbecco's Modified Eagle's Medium (DMEM)
Growth factors/hormones: Epidermal growth factor, insulin, hydrocortisone and other proprietary simulators of epidermal differentiation
Serum: None
Antibiotics: Gentamicin 5 μg/ml (10% of normal gentamicin level)
Anti-fungal agent: amphotericin B 0.25 μg/ml
pH indicator: phenol red

III. Tissue:
kit: standard Epiderm® kit (EPI-200) consists of 24 individual tissues, each 8 mm in diameter
Substrate: chemically modified, collagen-coated, 8 mm ID cell culture inserts are used
Culture: at air liquid interface
Histology: 8-12 cell layers (basal, spinous, and granular layers)
Stratum corneus: 10-15 layers (based on Transmission Electron Microscopy (TEM))
Length of experiments: cultures can be continued for up to 3 weeks with good retention of normal epidermal morphology. Cultures must be fed ebery other day with 5.0 ml of long life maintenance medium (EPI-100-LLMM), standard maintenance medium (EPI-100-MM), or new maintenance medium (EPI-100-NMM). Cell culture inserts are placed atop culture stands (MEL-STND) or washers (EPI-WSHR) in 6-well plates to allow use of 5.0 ml.

### Example 1

### Topically Oil-in-Water Emulsion

1.1. The Emulsion Composition is as Follows:

| Phase | N^{o} | Components | % (w/w) |
|---|---|---|---|
| Phase 0 | 01 | PEG 100 stearate (and) glyceryl stearate | 5.5 |
| | 02 | Paraffin liquid | 5 |
| | 03 | Caprylic/capric triglyceride | 2 |
| | 04 | phenoxyethanol | 0.9 |
| | 05 | Cetearyl alcohol | 2.5 |
| | 06 | dimethicone | 0.5 |
| | 07 | Tocopheryl acetate | 0.2 |
| | 08 | Ceramide 3 | 0.1 |
| | 09 | Sweet almond oil | 5 |
| Phase 1 | 10 | Water | 60 |
| | 11 | Acrylates C10-30 alkyl acrylate | 0.2 |
| | | crosspolymer | |
| | 12 | Potassium sorbate | 0.2 |
| | 13 | Glycerin | 3 |
| | 14 | Disodium EDTA | 0.1 |
| | 15 | Allantoin | 0.1 |
| Phase 2 | 21 | Xanthan gum | 0.4 |
| Phase 3 | 31 | Water | 5 |
| | 32 | Niacinamide | 2 |
| Phase 4 | 41 | Fucus Serratus Extract in glycerin | 0.5* |
| Phase 5 | 71 | perfume | 0.25 |
| Phase 6 | 61 | Water | q.s.p** 100 |
| | 62 | Triethamolamine (99%) | q.s.p** pH = 5.5 |

*It corresponds to 0.25% of pure Fucus Serratus algae and 0.25% of glycerin.
**“q.s.p” means “as needed for”.

1.2. The Process for Manufacturing the Oil-in-Water Emulsion is as Follows:

A. Preparation of Phases:

Phase 0: In an auxiliary reactor preheated at 80° C. components (01), (02), (03), (04), (05), (06), (07), (08) and (09) are added. The stirring is maintained until all components are totally melted.

Phase 1: In other reactor preheated at 80° C. component (11) is dispersed in component (10) by high stirring until total homogenization. Then, to the obtained dispersion, components (12), (13), (14) and (15) are sequentially added, maintaining the stirring conditions at 80° C.

Phase 3: In an auxiliary recipient component (32) is solubilized in component (31) by high stirring until a clear solution is obtained.

Phase 6: In an auxiliary recipient component (62) is solubilized in component (61) by high stirring until a clear solution is obtained.

B. Process for Manufacturing the Oil-in-Water Emulsion:

Step 1. In a reactor preheated at 80° C. to the above-obtained phase (1), component (21) is dispersed by moderate stirring maintaining the temperature until an aqueous homogenous gel is obtained.

Step 2. To the aqueous homogenous gel obtained in step 1, the above-obtained phase (0) is slowly added at 75-80° C. with stirring. Then, the emulsion thus obtained is homogenized at 3000 rpm during 10 min. After completing the homogenized time, the emulsion is cooled until 35° C. by moderate stirring. Step 3. To the above obtained emulsion of step 2, phase (3) is added at 40° C. The resultant emulsion is homogenized for 5 minutes and phase (4) and (5) is sequentially added at a temperature below 40° C. After 5 minutes, phase (6) is added in quantity sufficient to obtain a final pH 5.5 and it is homogenized.

### Example 2

### Synergistic Studies

The synergistic effect of the maturation grade of the cornified envelope of the Stratum corneum of a combination of glycerin, niacinamide and a glycerol extract of Fucus Serratus after being applied in an in-vitro model using the tissular system Epi-Derm® culture were tested. The tissular model Epi-Derm® has morphological and growing features similar to the human skin because Epi-Derm® is formed by stratum basale, stratum spinosum, stratum granulosum, and stratum corneum, and it is mitotic and metabolically active. Therefore, Epi-Derm® contains normal keratinocytes derived from human tissue (NHEK) which has been cultivated to form a multilayer model, highly differentiated, of the human epidermis, and corneocytes in the SC.

This synergistic test is based on the reduction of antigenicity to involucrin and the increase of hydrophobicity of mature CEr during its maturation process.

2.1. Preparation of Samples

A solution of glycerin and niacinamide (solution 1) in a proportion 1.2/1 (weight/volume), a solution of a glycerol extract of Fucus Serratus (solution 2), and a solution of glycerin, niacinamide, and a glycerol extract of Fucus Serratus in a proportion of 1.2/1/0.22 (weight/volume) (solution 3) in phosphate buffer saline (PBS) were prepared just previous their use.

The final concentrations of the active ingredients for their application on the surface of Epi-Derm® are as follow:

| | Concentrations | Concentrations |
|---|---|---|
| Active ingredients | (weight/volume) | (weight/weight) |
| Glycerin | 1.19% | 1.5% |
| Niacinamide | 1% | 1% |
| Extract of Fucus Serratus in | 0.22% | 0.25% |
| glycerin (1:1) | | |

2.1.1. Preparation of Solution 1

The process for preparing solution 1 was carried out by dissolving niacinamide in PBS at a concentration of 2% w/v, and diluting the above obtained solution with PBS at a proportion of 1:2.

Then, a mixture of 1 ml of the above-obtained solution of niacinamide in PBS with 1 ml of glycerin was topically applied on the surface of Epi-Derm®.

2.1.2. Preparation of Solution 3

The process for preparing solution 3 was carried out by dissolving 1 ml of solution 1 with 1 ml of the extract of Fucus Serratus, and diluting the above obtained solution with PBS at a proportion of 1:2.

Then, the resultant solution of the active ingredients was topically applied on the surface of Epi-Derm®.

2.2. Preparation of the Tissular System Epi-Derm®

Tissues of Epi-Derm® in a 6 well-plates in a preheated medium at 37° C. were incubated in an atmosphere with humidity at 37° C. and 5% of CO₂ overnight. After completing the incubation time, the medium of culture was removed and it was replaced by fresh medium.

2.3. Induction of Cutaneous Xerosis in the Incubated Tissues of Epi-Derm®

The test requires the induction of a cutaneous xerosis to the Epi-Derm® tissular system. To achieve this effect, 0.3 g of silica gel was added to the tissues obtained in section 2.2. After the addition, tissues were incubated in an atmosphere without humidity at 37° c. and 5% of CO₂ and in the presence of a recipient with silica gel for 24 hours.

2.4. Design of the Test

Solutions 1, 2 and 3 were separately applied to at least 5 tissues of Epi-Derm® according to the following experimental design:

| Group | Epi-Derm ® tissue | Treatment |
|---|---|---|
| 1 (normal skin blank) | Normal skin | No |
| 2 (dry skin blank) | Dry skin* | No |
| 3 | Dry skin* | Solution 1 |
| 4 | Dry skin* | Solution 2 |
| 5 | Dry skin* | Solution 3 |

*Dry skin induced according to the section 2.3.

2.5. Biological Method

Dry skin induced in tissues of Epi-Derm® were incubated with solution 1 (group 3), 2 (group 4) and 3 (group 5) separately for 24 hours. After completing the incubation time, a sample of the SC was obtained by the application of an adhesive tape on the surface of the tissue of each group.

Each adhesive tape was cut and 1 ml of an extracting solution containing 2% of docecylsulphate, 20 mM dithiothreitol, 5 mM ethylene diamine tetra ammonium salt (EDTA), and 0.1 M tris HCl (pH 8.5) was added. Cells were boiled at 100° C. for 10 minutes and after that the solution were centrifuged to 4000 g for 10 minutes. The precipitate obtained was recuperated and the extraction process was repeated until 3 times. The above obtained cells were re-suspended in a buffer solution. These suspensions were extended on the surface of a slide and were fixed with acetone at cold temperature (−30° C., 10 minutes).

Suspensions of cells of blank groups 1 and 2 were also obtained following the above mentioned process.

2.6. Analysis of the Results

The analysis consists in determining the percentage of mature CEr and immature CEf on the superficial layers of the SC to the Epi-Derm® system. Therefore, the grade of antigenicity of corneocytes on the superficial layers of the SC is evaluated by a tinction with anti-involucrin antibody followed by the addition of an anti-rabbit Immunoglobulin labeled with FITC; and the grade of hydrophobicity is evaluated by a tinction with Nile Red that is a selective fluorescent stain for intracellular lipid droplets.

The above-mentioned tinctions were carried out on slices of cells obtained in section 2.5. of groups 1-6 (section 2.4.). The samples thus obtained were observed with a fluorescent microscopy.

Mature CEr which are positives to the tinction with Nile Red were visualized as red cells while immature CEf which are positives to the tinction with FITC involucrin were visualized as green cells.

The number and percentages of the CEr and CEf detected in SC was summarized as follows:

Group 1: Normal skin blank

| tissue | N^{o} cells | n^{o} CEr | n^{o} CEf | % CEr | % CEf |
|---|---|---|---|---|---|
| 1 | 123 | 104.5 | 18.5 | 84.76 | 15.24 |
| 2 | 109.5 | 94 | 15.5 | 85.65 | 14.35 |
| 3 | 117.5 | 102 | 15.5 | 86.84 | 13.16 |
| 4 | 136.5 | 114 | 22.5 | 83.44 | 16.56 |
| 5 | 111 | 94 | 17 | 84.71 | 15.29 |
| media | 119.50 | 101.70 | 17.80 | 85.08 | 14.92 |
| Standard | 4.89 | 3.73 | 1.30 | 0.56 | 0.56 |
| error | | | | | |

Group 2: Dry skin blank

| tissue | N^{o} cells | n^{o} CEr | n^{o} CEf | % CEr | % CEf |
|---|---|---|---|---|---|
| 1 | 113.5 | 27 | 86.5 | 23.67 | 76.33 |
| 2 | 107.5 | 25 | 82.5 | 22.89 | 77.11 |
| 3 | 113 | 23 | 90 | 20.11 | 79.89 |
| 4 | 106.5 | 23 | 83.5 | 21.44 | 78.56 |
| 5 | 135 | 16 | 119 | 11.85 | 88.15 |
| media | 115.10 | 22.80 | 92.30 | 19.99 | 80.01 |
| Standard | 5.17 | 1.85 | 6.80 | 2.12 | 2.12 |
| error | | | | | |

Group 3: Dry skin treated with solution 1 (glycerin and niacinamide)

| tissue | N^{o} cells | n^{o} CEr | n^{o} CEf | % CEr | % CEf |
|---|---|---|---|---|---|
| 1 | 118 | 42 | 76 | 35.59 | 64.41 |
| 2 | 104 | 33 | 71 | 31.73 | 68.27 |
| 3 | 103 | 32 | 71 | 31.07 | 68.93 |
| 4 | 117 | 29 | 88 | 24.79 | 75.21 |
| 5 | 119 | 40 | 79 | 33.61 | 66.39 |
| media | 112.20 | 35.20 | 77.00 | 31.36 | 68.64 |
| Standard | 3.57 | 2.48 | 3.15 | 1.82 | 1.82 |
| error | | | | | |

Group 4: Dry skin treated with solution 2
(glycerol extract of Fucus Serratus)

| tissue | N^{o} cells | n^{o} CEr | n^{o} CEf | % CEr | % CEf |
|---|---|---|---|---|---|
| 1 | 104 | 33 | 71 | 31.73 | 68.27 |
| 2 | 120 | 38 | 82 | 31.67 | 68.33 |
| 3 | 133 | 43 | 90 | 32.33 | 67.67 |
| 4 | 136 | 53 | 83 | 38.97 | 61.03 |
| 5 | 115 | 43 | 72 | 37.39 | 62.61 |
| media | 121.60 | 42.00 | 79.60 | 34.42 | 65.58 |
| Standard | 5.89 | 3.32 | 3.59 | 1.56 | 1.56 |
| error | | | | | |

Group 5: Dry skin treated with solution 3 (glycerin, niacinamide and the
glycerol extract of Fucus Serratus)

| tissue | N^{o} cells | n^{o} CEr | n^{o} CEf | % CEr | % CEf |
|---|---|---|---|---|---|
| 1 | 106 | 79 | 27 | 74.53 | 25.47 |
| 2 | 122 | 102 | 20 | 83.61 | 16.39 |
| 3 | 121 | 90 | 31 | 74.38 | 25.62 |
| 4 | 100 | 82 | 18 | 82.00 | 18.00 |
| 5 | 108 | 84 | 24 | 77.78 | 22.22 |
| media | 111.40 | 87.40 | 24.00 | 78.46 | 21.54 |
| Standard | 4.33 | 4.07 | 2.35 | 1.89 | 1.89 |
| error | | | | | |

According to the ratio of mature CEr and immature CEf shown in control groups 1 and 2, it is established the reference values for CEr and CEf in normal skin and in dry skin. Thus, the reference values of normal skin are about 85% of mature CEr, and about 15% of immature CEf; and as reference values of dry skin are about 20% of mature CEr, and about 80% of immature CEf.

In summary, the results obtained in group 3, 4 and 5 are as follow:

| Group | Solutions | % Mature CEr | % Immature CEf |
|---|---|---|---|
| 3 | 1 | 31.36 (1.82) | 68.64 (1.82) |
| 4 | 2 | 34.42 (1.56) | 65.58 (1.56) |
| 5 | 3 | 78.46 (1.89) | 21.54 (1.89) |

Values between brackets indicate standard error (E.E.M).

As it is observed in the above summarized results, the percentage of mature CEr of group 5 where solution 3 is added (78.46%) is higher than the sum of the percentages of mature CEr of group 3 and group 4 (65.78%; 31.36%+34.42%=65.78%). Therefore, the synergistic effect of the combination of glycerin, niacinamide and the glycerol extract of Fucus Serratus (solution 3) in the promotion of the cornified envelope maturation in the SC of a dry skin is shown.

### PRIOR ART REFERENCE MENTIONED IN THE APPLICATION

1. EP 1374832
2. EP 1618867
3. A. V. Rawlings et al. “Moisturizer technology versus clinical performance”. Dermatologic Therapy, 2004, vol. 17, pp. 49-56.
4. N. Kitamura et al. “effect of niacinamide on the differentiation of human keratinocyte”, Journal of Dermatological Science, 1996, vol. 12, pp. 202.
5. A. V. Rawlings. “Trends in stratum corneum research and the management of dry skin conditions”. International Journal of Cosmetic Science. 2003, vol. 25, pp. 63-95.
6. A. V. Rawlings. “Stratum corneum moisturization at the molecular level: an update in relation to the dry skin cycle”, The Journal of Investigative Dermatology, 2005, vol. 124, pp. 1099-1110.
7. Hirao T. et al. “A novel non-invasive evaluation method of cornified envelope maturation in the stratum corneum provides a new insight for skin care cosmetics”. IFSCC Magazine, 2003, vol. 6, pp. 103.
8. Halzle E. “Effects of dermatitis, stripping, and steroids on the morphology of corneocytes. A new bioassay”, The journal of investigate dermatology, 1977, vol. 68(6), pp. 350-6.
9. Jane Fore-Pliger. “The epidermal skin barrier: Implications for the Wound Care Practitioner, Part I”, Advances in Skin Wound Care, 2004, vol. 17, pp. 417.
10. Contet-Audonneau J L, “A histological study of human wrinkle structures:
   comparison between sun-exposed areas of the face, with or without wrinkles, and sun-protected areas”, The British journal of dermatology, 1999, vol. 140(6), pp. 1038-47.
11. Academic press Dictionary of Science and Technology, 1992, pp. 531.
12. A terminological Dictionary of the Pharmaceutical Sciences. First edition, 2007, pp. 190.
13. T. Hirao, et al. “Identification of immature cornified envelopes in the barrier-impaired epidermis by characterization of their hydrophobicity and antigenicity of the components”. Experimental Dermatology, 2001, Vol. 10, pp. 35-44.

1. A combination comprising
glycerin,
niacinamide, and
an extract of Fucus Serratus.

2. The combination according to claim 1, wherein the extract of Fucus Serratus is a glycolic extract, wherein when the glycolic extract of Fucus Serratus is a glycerol extract, and the extract contains a part or the total amount of the glycerin of the combination.

3. The combination according to claim 2, wherein the glycolic extract of Fucus Serratus is a glycerol extract.

4. The combination according to claim 3, wherein the glycolic extract contains the total amount of the glycerin of the combination.

5. The combination according to claim 3, wherein the glycolic extract contains a part of the amount of the glycerin of the combination.

6. The combination according to claim 2, wherein the weight ratio (weight/weight/weight) between glycerin, niacinamide, and the glycolic extract of Fucus Serratus is comprised between 1.5:1:0.1 and 1.5:1:0.5.

7. The combination according to claim 6, wherein the weight ratio (weight/weight/weight) between glycerin, niacinamide, and the glycolic extract of Fucus Serratus is 1.5:1.0:0.2.

8. A topical pharmaceutical or cosmetic composition which comprises an effective amount of the combination as defined in claim 1, together with one or more appropriate topical pharmaceutically or cosmetically acceptable excipients or carriers.

9. The topical pharmaceutical or cosmetic composition according to claim 8, wherein the one or more appropriate topical pharmaceutically or cosmetically acceptable excipients or carriers is selected from the group consisting of a skin barrier recovery agent, a hydrating agent, an emollient, an emulsifier, a thickener, a humectant, a pH-regulating agent, an antioxidant, a preservative agent, a vehicle, and a mixture thereof.

10. The topical pharmaceutical or cosmetic composition according to claim 8, which is an emulsion.

11. The topical pharmaceutical or cosmetic composition according to claim 8, which is a surfactant base.

12. (canceled)

13. (canceled)

14. (canceled)

15. (canceled)

16. (canceled)

17. A method for the prophylaxis and/or treatment of a mammal suffering or is susceptible to suffer from a disease or condition which transcur through a deficit of maturation of the cornified envelope, wherein the disease or condition is selected from the group consisting of cutaneous xerosis, psoriasis, lamellar ichthyosis, skin aging, contact dermatitis, and atopic dermatitis, the method comprising administering to said mammal an effective amount of the topical pharmaceutical composition as defined in claim 8.

18. The method according to claim 17, wherein the disease or condition is cutaneous xerosis.

19. A method for skin care which comprises ameliorating at least one of the following symptoms: roughness, flakiness, dehydration, tightness, chapping, and lack of elasticity, the method comprising administering the topical cosmetic composition as defined in claim 8.

20. The method according to claim 19, wherein the topical cosmetic composition is a skin barrier recovery agent, or alternatively is a soothing agent.

21. The method according to claim 20, wherein the skin barrier recovery agent is a moisturizing agent.

22. The topical cosmetic composition according to claim 8, which is a moisturizing agent.

23. The topical cosmetic composition according to claim 8, which is a skin care agent.

24. The topical cosmetic composition according to claim 8, which is a soothing agent.
